SEQUENCE LISTING (17 sequences)

SEQ ID NO: 1 - protein, 19 aa, Homo sapiens
RGPSPSPVGSPASVAQSRS

SEQ ID NO: 2 - protein, 19 aa, Homo sapiens
RSPARTPPSEEDSAEAERL

SEQ ID NO: 3 - protein, 11 aa, Homo sapiens
RQPTPPFFGRD

SEQ ID NO: 4 - protein, 22 aa, Homo sapiens
RAGGGGGLGAGSPALSGGQGRR

SEQ ID NO: 5 - protein, 14 aa, Homo sapiens
RSCTPSPDQISHRA

SEQ ID NO: 6 - protein, 18 aa, Homo sapiens
KNSSNTSVGSPSNTIGRT

SEQ ID NO: 7 - protein, 21 aa, Homo sapiens
KEYIPGQPPLSQSSDSSPTRN

SEQ ID NO: 8 - protein, 12 aa, Homo sapiens
RFSSVSSPQPRS

SEQ ID NO: 9 - protein, 14 aa, Homo sapiens
RAQPSVSLGAPYRG

SEQ ID NO: 10 - protein, 14 aa, Homo sapiens
RAQPSASLGVGYRT

SEQ ID NO: 11 - protein, 14 aa, Homo sapiens
RNNSFTAPSTVGKR

SEQ ID NO: 12 - protein, 11 aa, Homo sapiens
RAQSLLSTDRE

SEQ ID NO: 13 - protein, 11 aa, Homo sapiens
RSSSFPYTTKG

SEQ ID NO: 14 - protein, 15 aa, Homo sapiens
RSCPSFSASSEGTRI

SEQ ID NO: 15 - protein, 14 aa, Homo sapiens
RNNSFTAPSTVGKR

SEQ ID NO: 16 - protein, 13 aa, Homo sapiens
RRTSFSTSDVSKL

SEQ ID NO: 17 - protein, 13 aa, Homo sapiens
RSQSYIPTSGCRA

(19) United States
(12) Patent Application Publication
(10) Pub. No.: US 2025/0116676 A1
(43) Pub. Date: Apr. 10, 2025

(54) PHOSPHOPEPTIDE/PHOSPHOPROTEIN SIGNATURE PREDICTING BASAL-LIKE BREAST CANCER RECURRENCE

(71) Applicants: THE HENRY M. JACKSON FOUNDATION FOR THE ADVANCEMENT OF MILITARY MEDICINE, INC., Bethesda, MD (US); WINDBER RESEARCH INSTITUTE, Windber, PA (US)

(72) Inventors: Praveen Kumar Raj KUMAR, Windber, PA (US); Hai HU, Windber, PA (US); Albert J. KOVATICH, Bethesda, MD (US); Craig D. SHRIVER, Bethesda, MD (US)

(21) Appl. No.: 18/907,207

(22) Filed: Oct. 4, 2024

Related U.S. Application Data
(60) Provisional application No. 63/587,785, filed on Oct. 4, 2023.

(51) Int. Cl. G01N 33/68; A61K 45/06; G16H 50/30

(52) U.S. Cl. CPC G01N 33/6893; A61K 45/06; G16H 50/30; G01N 2333/4704; G01N 2333/4706; G01N 2333/4712; G01N 2333/9015; G01N 2333/91215; G01N 2333/922

(57) ABSTRACT

The present disclosure provides a method of predicting basal-like breast cancer recurrence in a patient comprising (a) determining the phosphorylation status of at least one protein in a biological sample obtained from the patient; (b) identifying the patient as having a high risk of basal-like breast cancer recurrence if the phosphorylation status of the at least one protein is over-phosphorylated or under-phosphorylated as compared to a control; and (c) optionally administering to the patient a therapeutically effective amount of a chemotherapeutic if the patient is classified as having a high risk of basal-like breast cancer recurrence. Also disclosed herein are methods of treating a patient wherein the patient has been identified as having a high risk of basal-like breast cancer recurrence and kits for use in predicting basal-like breast cancer recurrence and/or prognosing basal-like breast cancer.

### CROSS-REFERENCE TO RELATED APPLICATION

This application claims priority to U.S. Provisional Application No. 63/587,785, filed 4 Oct. 2023, the contents of which are hereby incorporated by reference in its entirety.

### GOVERNMENT INTEREST

This invention was made with government support under W81XWH-12-2-0050 awarded by the United States Army Medical Research and Development Command and HU0001-16-2-0004 awarded by the Uniformed Services University of the Health Sciences. The government has certain rights in the invention.

### SEQUENCE LISTING

This application contains a Sequence Listing which has been submitted electronically in XML format and is hereby incorporated by reference in its entirety. Said XML copy, created on 4 Oct. 2024, is named HMJ-188-US_SL.xml and is 15,611 bytes in size.

### FIELD OF THE DISCLOSURE

The disclosure relates generally to phosphopeptide biomarkers, and more specifically to signatures developed using phosphopeptide biomarkers for use in predicting recurrence of basal-like breast cancer.

### BACKGROUND

Cancer is a leading cause of death worldwide, with the United States having an estimated more than 1,700,000 new cancer diagnoses and over 600,000 cancer fatalities in a single year. Breast cancer is the most common cancer diagnosis in women and the second-leading cause of cancer-related death among women. Major advances in cancer treatment, including breast cancer treatment, over the last 20 years, such as novel chemotherapeutics and other therapies, have led to significant improvement in the rate of survival. Despite the recent advances in cancer treatment, a significant number of patients will still ultimately die from recurrent disease. Thus, there is a need for clinicians to be able to predict the recurrence of a cancer based on the primary cancer of origin, so that treatment decisions can be made accordingly.

The identification of recurrence gene signatures having clinical utility is one option for the management and treatment of cancers. For example, Oncotype Dx® and MammaPrint® are commercially-available PCR and microarray assays that may be used to predict the risk of breast cancer recurrence, based on the expression of specific genes. Both Oncotype Dx® and MammaPrint®, however, which apply to early stage breast cancer cases, are limited to hormonal receptor positive and HER2 negative subtypes, with the latter further limited to patients under the age of 61, who have been diagnosed with lymph node-negative breast cancer and have a tumor size less than 5 cm. However, no such signature is currently available for triple-negative breast cancer (TNBC).

Another option for the management and treatment of cancers is the identification of phosphopeptide biomarkers. One post-translational modification of proteins is phosphorylation, i.e., the chemical attachment of a phosphate group to a specific amino acid residue of a protein, such as serine, threonine, and/or tyrosine residues. Aberration of phosphorylation by either over phosphorylation or under phosphorylation (dephosphorylation) of proteins has been associated with pathophysiological conditions, including cancer.

Therefore, identification of phosphopeptide biomarkers that are specific for recurrent cancers may provide more accurate diagnostic and/or prognostic potential needed in order to identify individuals who may be susceptible to a recurrence of basal-like breast cancer. Basal-like breast cancer comprises approximately 70% of TNBC cases.

### SUMMARY

Disclosed herein are methods of predicting basal-like breast cancer recurrence in a patient and methods of diagnosing or prognosing cancer in a patient. The expression levels of the phosphopeptide biomarkers disclosed herein can be used, for example, to predict the likelihood of a patient developing recurrent cancer, to help understand breast cancer development, or to inform treatment decisions. Also disclosed are methods of treating cancer in subject who have been subjected to the methods of predicting cancer recurrence or diagnostic/prognostic methods disclosed herein.

In certain embodiments, disclosed herein is a method of predicting basal-like breast cancer recurrence in a patient comprising (a) determining the phosphorylation status of at least one protein in a biological sample obtained from the patient, wherein the at least one protein is selected from ARID1A, SGTA, RBM14, RAB12, ZC3HAV1, CLASP1, EPRS, KIAA1522, PARN, PSMD11, FOXO3, DCK, MYO9B, or PLEKHA2; and (b) identifying the patient as having a high risk of cancer recurrence if the phosphorylation status of the at least one protein is (i) under-phosphorylated as compared to a control in at least one of the following locations: a serine at amino acid residue 696 of ARID1A, a threonine at amino acid residue 81 of SGTA, a threonine at amino acid residue 206 of RBM14, a serine at amino acid residue 21 of RAB12, a serine at amino acid residue 275 of ZC3HAV1, a serine at amino acid residue 1070 of CLASP1, or a serine at amino acid residue 886 of EPRS; or (ii) over-phosphorylated as compared to a control in at least one of the following locations: a serine at amino acid residue 339 of KIAA1522, a serine at amino acid residue 280 of RBM14, a serine at amino acid residue 256 of RBM14, a threonine at amino acid residue 498 of PARN, a serine at amino acid residue 14 of PSMD11, a serine at amino acid residue 413 of FOXO3, a serine at amino acid residue 11 of DCK, a serine at amino acid residue 496 of PARN, a serine at amino acid residue 1354 of MYO9B, or a serine at amino acid residue 184 of PLEKHA3. In certain embodiments, the methods disclosed herein further comprise administering to the patient a therapeutically effective amount of a cancer therapy if the patient is classified as having a high risk of cancer recurrence.

In certain aspects of the methods disclosed herein, the cancer is breast cancer, such as a basal-like breast cancer. In certain embodiments, the phosphorylation status of at least two, at least three, at least four, at least five, at least six, at least seven, at least eight, at least nine, at least ten, at least eleven, at least twelve, at least thirteen, or all fourteen of the proteins is determined.

In certain embodiments, the at least one protein is selected from ARID1A, SGTA, RBM14, RAB12, ZC3HAV1, CLASP1, or EPRS, and at least one, at least two, at least three, at least four, at least five, at least six, or seven of the proteins are under-phosphorylated as compared to a control. In certain embodiments, the at least one protein is selected from KIAA1522, RBM14, PARN, PSMD11, FOXO3, DCK, MYO9B, or PLEKHA3, and at least one, at least two, at least three, at least four, at least five, at least six, at least seven, or eight of the proteins are over-phosphorylated as compared to a control.

In certain aspects of the methods disclosed herein, the method further comprises obtaining from the patient a biological sample comprising cancer tissues or cells.

Also disclosed herein are methods of treating a cancer patient, the method comprising administering to a patient a therapeutically effective amount of a chemotherapeutic, wherein the patient has been identified as having a high risk of cancer recurrence according to the methods of predicting cancer recurrence disclosed herein. In certain embodiments, the cancer therapy is one or more of surgery, radiation therapy, hormone therapy, chemotherapy, biological therapy, or high intensity focused ultrasound. In certain embodiments, the control comprises control tissues or cells obtained from tissues or other biological samples or the phosphorylation status obtained from control tissues or cells, such as tissues or cells obtained from a patient or pool of patients who exhibited non-recurrent cancer or non-cancerous cells, such as tissues or cells obtained from a patient or pool of patients who are cancer-free, and in certain embodiments, the control comprises a standard or reference that reflects the phosphorylation status of phosphopeptides in a sample or pool of samples known to contain non-recurrent cancer or known to be cancer-free, such as might be part of an electronic database or computer program.

In certain embodiments, the phosphorylation status of a phosphopeptide signature is determined by calculating a recurrence index score for the phosphopeptide signature. In certain embodiments, the recurrence index is calculated as the sum of the weights calculated for each phosphopeptide in the phosphopeptide signature, including, for example, using Formula 1, as described herein. In certain embodiments, the raw recurrence index obtained using Formula 1 is further scaled, including, for example, using Formula 2, as described herein.

Also disclosed herein are kits for use in predicting cancer recurrence and/or prognosing cancer recurrence. In certain embodiments, the kit comprises a plurality of probes for detecting a phosphophorylation status of at least 1, such as at least 2, at least 3, at least 4, at least 5, at least 6, at least 7, at least 8, at least 9, at least 10, at least 11, at least 12, at least 13, at least 14, at least 15, at least 16, or 17 of the following phosphorylation sites: a serine at amino acid residue 339 of KIAA1522, a serine at amino acid residue 280 of RBM14, a serine at amino acid residue 256 of RBM14, a threonine at amino acid residue 498 of PARN, a serine at amino acid residue 14 of PSMD11, a serine at amino acid residue 413 of FOXO3, a serine at amino acid residue 11 of DCK, a serine at amino acid residue 496 of PARN, a serine at amino acid residue 1354 of MYO9B, a serine at amino acid residue 184 of PLEKHA3; a serine at amino acid residue 696 of ARID1A, a threonine at amino acid residue 81 of SGTA, a threonine at amino acid residue 206 of RBM14, a serine at amino acid residue 21 of RAB12, a serine at amino acid residue 275 of ZC3HAV1, a serine at amino acid residue 1070 of CLASP1, or a serine at amino acid residue 886 of EPRS, wherein the plurality of probes contains probes for detecting no more than 500 different phosphopeptides.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced.

FIG. 1A is a map showing K-means consensus clustering of 50 tumors using consistently quantified 245 phosphopeptides, as described in Example 1.

FIG. 1B shows Kaplan-Meier curves of time to disease progression in years for four phosphoproteome clusters (Basal 1, Basal 2, Her2 enriched, and LumA enriched) for the end point of progression-free interval (PFI), as described in Example 1.

FIG. 1C is a map showing the Hierarchical clustering of the 16 Basal-like tumors using 76 significantly (FC>1.2 for upregulated, FC<1/1.2 for downregulated, and FDR≤0.2) differentially expressed phosphopeptides between 10 Basal cases in the Basal 2 cluster and 6 Basal cases in the Basal 1 cluster, as described in Example 1.

FIG. 1D shows Kaplan-Meier curves of time to disease progression in years for Basal cases in the two Basal clusters (Basal 1 and Basal 2) for PFI, as described in Example 1. P-values and the number of events/number of cases are given in the plot legends.

FIG. 2A shows Kaplan-Meier curves of time to disease progression in years for the significantly (FC>1.2 for upregulated, FC<1/1.2 for downregulated, and FDR≤0.2) differentially expressed phosphopetides between 10 Basal cases of the Basal_2 cluster (high relapse-risk) and 6 Basal cases of the Basal_1 cluster (low relapse-risk), showing high (>median) expression of 10 up-regulated phosphopeptides in the Basal_2 cluster that were significantly (p<0.05) associated with a worse progression free interval (PFI), as described in Example 1. Gene name, P-value, phosphosite and the number of events ‘/’ number of cases are given in each plot.

FIG. 2B shows Kaplan-Meier curves of time to disease progression in years for the significantly (FC>1.2 for upregulated, FC<1/1.2 for downregulated, and FDR≤0.2) differentially expressed phosphopetides between 10 Basal cases of the Basal_2 cluster (high relapse-risk) and 6 Basal cases of the Basal_1 cluster (low relapse-risk), showing low (<median) expression of 7 down-regulated phosphopeptides in the Basal_2 cluster that were significantly (p<0.05) associated with a worse progression free interval (PFI), as described in Example 1. Gene name, P-value, phosphosite and the number of events ‘/’ number of cases are given in each plot.

The drawings are not necessarily to scale, and may, in part, include exaggerated dimensions for clarity.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order that the present embodiments may be more readily understood, certain terms are first defined. Additional definitions are set forth throughout the detailed description.

The term “detecting” or “detection” means any of a variety of methods known in the art for determining the presence or amount of a nucleic acid or a protein. As used throughout the specification, the term “detecting” or “detection” includes either qualitative or quantitative detection.

The term “non-recurrent cancer sample” refers to a cancer sample from a patient who did not experience cancer recurrence in a given amount of time after treatment. In certain embodiments, a non-recurrent cancer sample is a cancer sample from a patient who did not experience a cancer recurrence for at least 2 years after treatment, such as at least 3 years, at least 4 years, at least 5 years, 6 years, at least 7 years, at least 8 years, or at least 9 years after treatment.

The terms “prognosis” and “prognosing” as used herein mean predicting the likelihood of death from the cancer and/or recurrence or metastasis of the cancer within a given time period, with or without consideration of the likelihood that the cancer patient will respond favorably or unfavorably to a chosen therapy or therapies.

The term “isolated,” when used in the context of a polypeptide or nucleic acid refers to a polypeptide or nucleic acid that is substantially free of its natural environment and is thus distinguishable from a polypeptide or nucleic acid that might happen to occur naturally. For instance, an isolated polypeptide or nucleic acid is substantially free of cellular material or other polypeptides or nucleic acids from the cell or tissue source from which it was derived.

The terms “polypeptide,” “peptide,” and “protein” are used interchangeably herein to refer to polymers of amino acids.

The term “polypeptide probe” as used herein refers to a labeled (e.g., isotopically labeled) polypeptide that can be used in a protein detection assay (e.g., mass spectrometry) to quantify a polypeptide of interest in a biological sample.

In the specification, the term “sample” should be understood to mean tumor cells, tumor tissue, non-tumor tissue, conditioned media, blood or blood derivatives (serum, plasma, etc.), urine, or cerebrospinal fluid.

In the specification, the term “recurrence” should be understood to mean the recurrence of the cancer which is being sampled in the patient, in which the cancer has returned to the sampled area after treatment, for example, if sampling breast cancer, recurrence of the breast cancer in the (source) breast tissue. The term should also be understood to mean recurrence of a primary cancer whose site is different to that of the cancer initially sampled, that is, the cancer has returned to a non-sampled area after treatment, such as non-locoregional recurrences.

As used herein, the term “recurrence index” or “recurrence index score” refers to a numerical index calculated as a weighted linear combination of the expression levels of the phosphopeptides in a phosphopeptide signature as disclosed herein, such as a 17-phosphopeptide signature (or subsets of phosphopeptides within the phosphopeptide signature). In certain embodiments, the weight in the weighted linear combination calculated for each phosphopeptide represents the importance of a phosphopeptide's contribution to the prediction of cancer recurrence, and the recurrence index may be calculated as disclosed herein.

Disclosed herein are differentially expressed phosphopeptides that may be used to identify patients at an increased risk for recurrent basal-like breast cancer.

The present disclosure is based on the discovery that certain phosphopeptides are differentially expressed in basal-like cases and can be used to identify cases of high-relapse risk and low relapse-risk basal-like breast cancer, with a significant difference in progression free interval of surviving cases. Phosphopeptide biomarkers are identified in Table A and Table B below and may account for all or part of a phosphopeptide signature. In certain embodiments, the differentially expressed phosphopeptide biomarkers in Table A and Table B represent those that are up-regulated (over-phosphorylated or increased phosphorylation) and down-regulated (under-phosphorylated or decreased phosphorylation) at a particular amino acid residue or residues in high-relapse risk basal-like breast cancer compared to low-relapse risk cases.

In a first aspect, disclosed here is a method of predicting basal-like breast cancer recurrence in a patient. In certain embodiments, the method comprises (a) determining the phosphorylation status of at least one phosphopeptide that is differentially expressed between high-relapse-risk and low-relapse-risk basal-like breast cancer tumors in the patient; (b) classifying the patient as at-risk or not-at-risk for cancer recurrence based on the phosphorylation status of the at least one phosphopeptide that is differentially expressed; and (c) optionally administering to the patient a therapeutically effective amount of a cancer therapy if the patient is classified as being at-risk for cancer recurrence.

In certain embodiments, the phosphorylation status of at least one phosphopeptide may be determined by any means known in the art, including, for example, immunohistochemistry, a protein array, and/or mass spectrometry techniques. In certain embodiments, the phosphorylation status of a phosphopeptide signature may be determined by calculating a recurrence index score for the phosphopeptide signature.

As disclosed herein, the recurrence index may be calculated as the sum of the weights calculated for each phosphopeptide in the phosphopeptide signature. For example, in certain embodiments the recurrent index may be calculated using the following Formula 1:

$$R I = {\sum }_{i = 1}^{17} {w}_{i} {log}_{2} {x}_{i} \qquad Formula 1$$

wherein the raw recurrence index (RI) is calculated by the weighted linear combination of log 2 transformed normalized assay results of phosphopeptides x, with t-statistic w, for each phosphopeptide serving as the weight. Descriptions of the 17 phosphopeptides are provided in Table A and Table B below, as well as exemplary peptide fragments that may be used to identify the phosphorylation status of the phosphopeptide and the weights of the 17 phosphopeptides, as calculated using Formula 1.

In certain embodiments, the raw recurrence index score may subsequently be scaled to 0-10 using a transformation formula as set forth in Formula 2 below:

$$R I . {scale}_{i} = \frac{max ( R I ) - R {I}_{i}}{max ( R I ) - min ( R I )} \times 10 \qquad Formula 2$$

A score that is above a threshold indicates a higher chance of recurrence.

In another aspect, disclosed herein is a method of treating a patient with basal-like breast cancer. In certain embodiments, the method comprises administering to the patient a therapeutically effective amount of a cancer therapy if the patient is classified as being at-risk for cancer recurrence according to the methods disclosed herein.

In certain embodiments, DNA, RNA, and/or proteins may be obtained from a tissue sample, such as a tumor, by any means known in the art and analyzed by any means known in the art. In certain embodiments, the protein may be first digested to produce peptides, and the protein and/or peptide may be analyzed by mass spectrometry. In some embodiments, the protein and/or peptide may be purified, such as by column purification, optionally dried, and fractionated. In some embodiments, one or more fractions may be enriched based on at least one post-translational modification, such as phospho-enrichment by affinity chromatography and/or binding, ion exchange chromatography, chemical derivatization, immunoprecipitation, co-precipitation, or a combination thereof. In certain embodiments, the phospho-enriched fractions may be subject to analysis, such as by mass spectrometry.

### Phosphopeptide Biomarkers

As disclosed herein, various phosphopeptide biomarkers have been identified, the differential phosphorylation of which (i.e., over- or under-phosphorylation) at various amino acid residues as compared to a control may be used to identify a sample as comprising basal-like breast cancer and to classify the cancer according to the risk of recurrence. Table A below sets forth 10 phosphopeptides (or nucleic acids encoding the phosphopeptides) that may be over-phosphorylated in high-relapse risk basal-like breast cancer as compared to low-relapse risk basal-like breast cancer, wherein the * in Table A indicates the site of the over-phosphorylated amino acid residue. Table B below sets forth 7 phosphopeptides (or nucleic acids encoding the phosphopeptides) that may be under-phosphorylated in high-relapse risk basal-like breast cancer as compared to low-relapse risk basal-like breast cancer, wherein the * in Table A indicates the site of the under-phosphorylated amino acid residue.

TABLE A
Over-phosphorylated Phosphopeptides in High Relapse Risk

| | NCBI Ref. Seq./ | Phosphoryl- | | | |
|---|---|---|---|---|---|
| Gene | Ensembl Protein | ation site | | | |
| Symbol | ID | (p-value) | Description | Peptide | Weight |
| KIAA1522 | NM_001198972.2/ | S339s | KIAA1522 | RFSS*VSSPQP | 2.90 |
| | ENSP00000362579/ | (0.0213) | (aka NHSL3) | RS | |
| | ENST00000373480.1 | | | (SEQ ID NO: 8) | |
| RBM14 | NM_006328.4/ | S280s | RNA- | RAQPSVS*LG | 5.43 |
| | ENSP00000311747/ | (0.0449) | binding motif | APYRG | |
| | ENST00000310137.5 | | protein 14 | (SEQ ID NO: 9) | |
| RBM14 | NM_006328.4/ | S256s | RNA- | RAQPSAS*LG | 4.66 |
| | ENSP00000311747/ | (0.0449) | binding motif | VGYRT | |
| | ENST00000310137.5 | | protein 14 | (SEQ ID NO: | |
| | | | | 10) | |
| PARN | NM_002582.4/ | T498t | Poly-A | RNNSFT*APS | 4.58 |
| | ENSP00000345456/E | (0.0449) | specific | TVGKR | |
| | NST00000341484.11 | | ribonuclease | (SEQ ID NO: | |
| | | | | 11) | |
| PSMD11 | NM_002815.4/ | S14s | Proteosome | RAQS*LLSTD | 3.93 |
| | ENSP00000261712/ | (0.0449) | 26S Subunit, | RE | |
| | ENST00000261712.8 | | non-ATPase | (SEQ ID NO: | |
| | | | 11 | 12) | |
| FOXO3 | NM_001415139.1/ | S413s | Forkhead | RSSS*FPYTT | 3.82 |
| | ENSP00000339527/ | (0.0449) | box O3 | KG | |
| | ENST00000343882.10 | | | (SEQ ID NO: | |
| | | | | 13) | |
| DCK | NM_000788.3/ | S11s | Deoxycytidine | RSCPS*FSASS | 3.29 |
| | ENSP00000286648/ | (0.0449) | kinase | EGTRI | |
| | ENST00000286648.10 | | | (SEQ ID NO: | |
| | | | | 14) | |
| PARN | NM_002582.4/ | S496s | Poly-A | RNNS*FTAPS | 3.13 |
| | ENSP00000345456/ | (0.0449) | specific | TVGKR | |
| | ENST00000341484.11 | | ribonuclease | (SEQ ID NO: | |
| | | | | 15) | |
| MYO9B | NM_001130065.2/ | S1354s | Myosin IXB | RRTS*FSTSD | 3.10 |
| | ENSP00000380444/ | (0.0449) | | VSKL | |
| | ENST00000397274.6 | | | (SEQ ID NO: | |
| | | | | 16) | |
| PLEKHA2 | NM_021623.2/ | S184s | Pleckstrin | RSQS*YIPTSG | 2.75 |
| | ENSP00000393860.1 | (0.0449) | homology | CRA | |
| | | | domain | (SEQ ID NO: | |
| | | | containing | 17) | |
| | | | A2 | | |

TABLE B
Under-phosphorylated Phosphopeptides in High Relapse Risk

| | NCBI Ref. Seq./ | Phosphoryl- | | | |
|---|---|---|---|---|---|
| | Ensembl Protein | ation site | | | |
| Symbol | ID | (p-value) | Description | Peptide | Weight |
| ARID1A | NM_139135.4/ | S696s | AT-rich | RGPS*PSPVGSP | −3.18 |
| | ENSP00000320485.13 | (0.0213) | interaction | ASVAQSRS | |
| | | | domain 1A | (SEQ ID NO: 1) | |
| SGTA | NM_003021.4/ | T81t | Small | RSPART*PPSEE | −5.90 |
| | ENSP00000221566.7 | (0.0449) | glutamine rich | DSAEAERL | |
| | | | tetratricopeptide | (SEQ ID NO: 2) | |
| | | | repeat co- | | |
| | | | chaperone alpha | | |
| RBM14 | NM_006328.4/ | T206t | RNA-binding | RQPT*PPFFGR | −2.88 |
| | ENSP00000311747.5 | (0.0449) | motif protein | D | |
| | | | 14 | (SEQ ID NO: 3) | |
| RAB12 | NM_001025300.3/ | S21s | RAB12, | RAGGGGGLGA | −2.53 |
| | ENSP00000331748.6 | (0.0449) | member RAS | GS*PALSGGQG | |
| | | | oncogene family | RR | |
| | | | | (SEQ ID NO: 4) | |
| ZC3HAV1 | NM_020119.4/ | S275s | Zinc finger | RSCTPS*PDQIS | −2.52 |
| | ENSP00000242351.10 | (0.0449) | CCCH-type | HRA | |
| | | | containing, | (SEQ ID NO: 5) | |
| | | | antiviral 1 | | |
| CLASP1 | NM_015282.3/ | S1070s | Cytoplasmic | KNSSNTSVGS* | −2.50 |
| | ENSP00000380717.4 | (0.0449) | linker | PSNTIGRT (SEQ | |
| | ENSP00000380717.5 | | associated | ID NO: 6) | |
| | ENSP00000380717.6 | | protein 1 | | |
| | ENSP00000380717.7 | | | | |
| EPRS | NM_004446.3/ | S886s | Glutamyl- | KEYIPGQPPLS | −2.41 |
| | ENSP00000355890.8 | (0.0449) | prolyl-tRNA | QSSDSS*PTRN | |
| | | | synthetase 1 | (SEQ ID NO: 7) | |

Information related to each protein provided in Tables A and B, including their amino acid and nucleic acid sequences, are accessible via publicly available databases using the respective NCBI (“NM”) and Ensembl Reference Nos. provided in Tables A and B, including for example through the NCBI website at ncbi.nlm.nih.gov/nuccore/or the Ensembl website at ensembl.org/Homo_sapiens/Info/Index.

In some embodiments, the phosphorylation status of a phosphopeptide biomarker is determined by determining the post-translational status (e.g., phosphorylation status) of the phosphopeptide biomarker, such as determining the presence or absence of a phosphate at one or more particular amino acid location on the phosphopeptide biomarker. Phosphorylation status may be determined by any method known in the art, including, for example, by mass spectrometry. In certain embodiments, the patient has been diagnosed with basal-like breast cancer, and the phosphorylation status of at least one of the phosphopeptide biomarkers may be used to prognose the risk of the basal-like breast cancer recurring in the patient.

Many of the 14 genes identified above in Tables A and B have been previously reported to play a role in breast cancer and other cancers. Among the 10 phosphopeptides up-regulated in the high relapse-risk group, KIAA1522, DCK, FOXO3 and MYO9B have been associated with aggressive cancer phenotypes. KIAA1522's elevation in triple-negative breast cancer tissues, for example, has been reported for its oncogenic potential and role in promoting visceral metastasis. The DCK gene, known for its increased expression in breast cancers with poor prognosis, is associated with the action of Decitabine, an FDA-approved drug for certain blood cancers, which has also been shown to inhibit the growth of triple-negative breast cancer. FOXO3 has been implicated in the coordinated increases in glycolysis and apoptosis resistance in TNBC and proposed as an attractive therapeutic target for TNBC. High levels of MYO9B have been shown to promote actin reorganization by reducing filaments and to stimulate metastasis by breaking down stress fibers and reducing cell adhesion, thereby enhancing the cancer phenotype in both prostate and lung cancer.

The down-regulation of phosphopeptides in genes like ARID1A, EPRS, and ZC3HAV1 in the high relapse-risk breast cancer group may offer insights into their roles as tumor suppressors and regulatory molecules. The downregulation of ARID1A, known for its potential in DNA repair and immune response modulation, in triple-negative breast cancer, marks it as a target for immune checkpoint inhibitors. Additionally, EPRS has been reported as a regulator of cell proliferation and estrogen signaling in ER+breast cancer and has also been implicated as a potential treatment target for basal-like breast cancer. ZC3HAV1, a PARP family enzyme, promotes proliferation and metastasis by regulating KRAS in pancreatic cancer and is involved in facilitating DNA repair and promoting tumorigenesis in breast cancer.

Notably, three phosphopeptides from RBM14 were identified with different directions of differential expression. RBM14 is known to function in transcription and RNA splicing; different isoforms are encoded by alternatively spliced transcript variants and have been reported to have opposing effects on transcription. The different directions of enrichment of the three RBM14 phosphopeptides indicate that there may be coordinated or opposing regulation among the different phosphorylation sites to carry out the different functions of this protein. RBM14 is known to physically interact with PARP1, which is a key player in the DNA damage response (DDR) network and a target of cancer therapy. RBM14 has also been implicated in the migration of breast cancer, heightened radio-resistance in glioblastoma, and more recently, promoting cell growth in lung cancer.

### Detecting Protein Expression or Phosphorylation Status Thereof

As used herein, measuring or detecting the phosphorylation status of a phosphopeptide biomarker is determined by determining the post-translational status (e.g., phosphorylation status) of the phosphopeptide biomarker, such as determining the presence or absence of a phosphate at one or more particular amino acid location on the phosphopeptide biomarker. Phosphorylation status may be determined by any method known in the art, including, for example, by mass spectrometry. It may also be possible to detect a phosphorylated peptide or protein using an antibody that specifically binds to the phosphorylated or unphosphorylated phosphopeptide biomarkers, or another type of immunoassay to measure the level of a protein or phosphopeptide of interest.

Several methods and devices are known for determining levels of proteins including immunoassays, such as described, for example, in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays may include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays may include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO 2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

One type of immunoassay, called nucleic acid detection immunoassay (NADIA), combines the specificity of protein antigen detection by immunoassay with the sensitivity and precision of the polymerase chain reaction (PCR). This amplified DNA-immunoassay approach is similar to that of an enzyme immunoassay, involving antibody binding reactions and intermediate washing steps, except the enzyme label is replaced by a strand of DNA and detected by an amplification reaction using an amplification technique, such as PCR. Exemplary NADIA techniques are described in U.S. Pat. No. 5,665,539 and published U.S. Application 2008/0131883, both of which are hereby incorporated by reference in their entirety. Briefly, NADIA uses a first (reporter) antibody that is specific for the protein of interest and labelled with an assay-specific nucleic acid. The presence of the nucleic acid does not interfere with the binding of the antibody, nor does the antibody interfere with the nucleic acid amplification and detection. Typically, a second (capturing) antibody that is specific for a different epitope on the protein of interest is coated onto a solid phase (e.g., paramagnetic particles). The reporter antibody/nucleic acid conjugate is reacted with sample in a microtiter plate to form a first immune complex with the target antigen. The immune complex is then captured onto the solid phase particles coated with the capture antibody, forming an insoluble sandwich immune complex. The microparticles are washed to remove excess, unbound reporter antibody/nucleic acid conjugate. The bound nucleic acid label is then detected by subjecting the suspended particles to an amplification reaction (e.g. PCR) and monitoring the amplified nucleic acid product.

Although immunoassays have been used for the identification and quantification of proteins, recent advances in mass spectrometry (MS) techniques have led to the development of sensitive, high-throughput MS protein analyses. The MS methods can be used to detect low abundant proteins in complex biological samples. For example, it is possible to perform targeted MS by fractionating the biological sample prior to MS analysis. Common techniques for carrying out such fractionation prior to MS analysis include, for example, two-dimensional electrophoresis, liquid chromatography, and capillary electrophoresis. Selected reaction monitoring (SRM), also known as multiple reaction monitoring (MRM), has also emerged as a useful high-throughput MS-based technique for quantifying targeted proteins in complex biological samples.

### Samples

The methods described herein involve analysis of phosphorylation status in peptides from biological samples obtained from a cancer patient. Cancer cells may be found in a biological sample, such as a tumor, a tissue, or blood. Proteins or polypeptides may be isolated from the sample prior to detecting phosphorylation status. In one embodiment, the biological sample comprises tumor tissue and is obtained through a biopsy. In certain embodiments, the sample is obtained through laser microdissection. The methods disclosed herein can be used with biological samples collected from a variety of mammals, and in certain embodiments, the methods disclosed herein may be used with biological samples obtained from a human subject. In certain embodiments, the samples may be fresh, and in certain embodiments, the samples may be frozen. In certain embodiments, the samples may be fixed-formalin paraffin-embedded (FFPE) tissue samples.

### Controls

In certain embodiments, the control may be any suitable reference that allows evaluation of the phosphorylation status of the peptides in the biological sample as compared to the phosphorylation status of the same peptides in a sample comprising control tissues or cells or peptides obtained from the control tissues or cells. In certain embodiments, the control tissues or cells may be non-recurrent cancerous tissues or cells, such as tissues or cells obtained from a patient or pool of patients who exhibited non-recurrent cancer. In certain embodiments, the control tissues or cells may be non-cancerous tissues or cells, such as tissues or cells obtained from a patient or pool of patients who are cancer-free. Thus, for instance, the control can be a sample that is analyzed simultaneously or sequentially with the test sample, or the control can be the average phosphorylation status of the phosphopeptides of interest in a pool of samples known to be non-recurrent cancer or known to be cancer-free. In certain embodiments, the control can be embodied, for example, in a pre-prepared microarray used as a standard or reference, or in data that reflects the phosphorylation status of relevant phosphopeptides in a sample or pool of samples known to contain non-recurrent cancer or known to be cancer-free, such as might be part of an electronic database or computer program.

### Cancer Types and Staging

In various embodiments, the cancer may be selected from testicular, prostate, colorectal, breast, pancreatic, ovarian, cervical, uterine, bone (e.g., osteosarcoma, chondrosarcoma, Ewing's tumor, and chordoma), bladder, skin (e.g., melanoma, squamous cell carcinoma and basal cell carcinoma), blood (e.g., leukemia, lymphoma, and myeloma), lung (e.g., squamous cell carcinoma, adenocarcinoma, large cell carcinoma, small cell carcinoma, and carcinoid tumors), central nervous system, and kidney cancer. In certain embodiments, the cancer is selected from a basal-like subtype breast cancer.

In certain embodiments, the cancer is breast cancer. When diagnosing breast cancer, breast tumors may be classified based on hormone receptor status, such as estrogen receptor (ER), progesterone receptor (PR), and human epidermal growth factor receptor-2 (HER2). Accordingly, the cancer may be characterized as ER+ or ER−, PR+ or PR−, and HER2+ or HER2− (and combinations thereof). Additionally, breast tumors may be classified based on various gene expression features, including luminal A, luminal B, Her2− enriched, basal-like, and normal-like. As known to those of ordinary skill in the art, the basal-like subtype largely overlaps with the “triple negative” subtype (i.e., ER−, PR−, and HER2− based on immunohistochemistry assays of these protein receptors), it being understood that not all basal-like subtype breast cancers are triple negative, and not all triple-negative breast cancers are of the basal-like subtype. As used herein, the basal-like breast cancer mostly, but not exclusively, includes ER−, PR− and HER2−, whereas the luminal subtype is mostly ER+. The breast cancer subtypes may be associated with distinct biological features and clinical prognosis and may be assigned, for example, based on the expression of a panel of 50 genes to predict breast cancer subtypes. See Parker, et al., Supervised Risk Predictor of Breast Cancer Based on Intrinsic Subtype, J. Clin. Oncol. 2009 Mar. 10; 27(8):1160-7.

Many cancers, including breast cancers, may be further diagnosed and classified based on the TNM staging system. In the TNM staging system, a tumor stage (T stage), lymph node stage (N stage) and metastases stage (M stage) can be assessed. As used herein, T0 indicates no evidence of tumor; T1 indicates the tumor is less than or equal to 2 cm; T2 indicates the tumor is greater than 2 cm but less than or equal to 5 cm; T3 indicates the tumor is greater than 5 cm; and T4 indicates a tumor of any size growing in the wall of the breast or skin, or inflammatory breast cancer. For lymph node staging, NO indicates the cancer is not present in any regional lymph nodes; N1 indicates the cancer has spread to 1 to 3 axillary lymph nodes or to one internal mammary lymph node; N2 indicates the cancer has spread to 4 to 9 axillary lymph nodes or to multiple internal mammary lymph nodes; and N3 indicates the cancer has spread to 10 or more axillary lymph nodes, the cancer has spread to the infraclavicular or supraclavicular lymph nodes, the cancer has spread to the internal mammary lymph nodes, or the cancer affects 4 or more axillary lymph nodes and minimum amounts of cancer are in the internal mammary nodes or in sentinel lymph node biopsy. For metastasis staging, M0 indicates there is no spread of the cancer outside of the site of origin, and M1 indicates there is spread to at least one distant organ.

Based on the TNM staging, a cancer may be staged in a range of 0 to IV, wherein stage IV indicates the cancer has metastases; in general, the higher the stage, the poorer the prognosis. Thus, cancers with a high stage (Stage III and Stage IV) have a poorer prognosis for overall survival than cancers with a lower stage (Stage I and Stage II). In general, the lower the stage, the less aggressive the cancer and the better the prognosis (outlook for cure or long-term survival). The higher the stage, the more aggressive the cancer and the poorer the prognosis for long-term, metastases-free survival.

Cancer may also be graded on a scale of G1 to G4, wherein the higher the grade, the more likely the cancer is to grow and spread. G1 indicates that the cells of the biopsied cancerous tissue are well-differentiated, i.e., appear more like the cells of the tissue of origin (e.g., breast or ovarian tissue), and therefore less likely to spread, and G2 indicates that the cells of the biopsied cancerous tissue are moderately differentiated. G3 and G4 indicate that the cells of the biopsied cancerous tissue are poorly differentiated, and therefore the most likely to spread.

In certain embodiments, the phosphopeptide biomarkers disclosed herein can be used to diagnose or prognose cancer, or to predict cancer recurrence, such as recurrence of a basal-like breast cancer.

### Patient Treatment

Disclosed herein are methods of diagnosing, prognosing, and predicting a cancer, including recurrence of cancer, in a sample obtained from a patient, in which the phosphorylation status of at least one phosphopeptide biomarker in tumor cells and/or tissues is analyzed. If a sample shows over-phosphorylation or under-phosphorylation of certain amino acids in a phosphopeptide biomarker or multiple phosphopeptide biomarkers relative to a control, then there is an increased likelihood that the patient's cancer will recur and/or have a worse prognosis than if the sample does not show differential phosphorylation status relative to a control. Thus, the methods of detecting or prognosing cancer may be used to assess the need for therapy or to monitor a response to a therapy (e.g., disease-free recurrence following surgery or other therapy). In the event of such a result, the methods of prognosing cancer may include one or more of the following steps: informing the patient that they are likely to have a cancer recurrence; and treating the patient by an appropriate cancer therapy.

In certain embodiments of the methods disclosed herein, if the patient is predicted to have a high risk of cancer recurrence, an appropriate cancer therapy may be more aggressive than if the patient is predicted to have a low risk of cancer recurrence.

Cancer treatment options include surgery, radiation therapy, hormone therapy, chemotherapy, biological therapy, and/or high intensity focused ultrasound. Drugs approved for cancer are known to the ordinarily skilled artisan based on the cancer type and grade. Thus a method as described herein may, after a positive result, include a further treatment step, such as, surgery, radiation therapy, hormone therapy, chemotherapy, biological therapy, or high intensity focused ultrasound.

Drugs for cancer treatment include, but are not limited to: melphalan (ALKERAN®), bevacizumab (ALYMSYS®, AVASTIN®, MVASI®, ZIRABEV®), carboplatin (PARAPLATIN®), cisplatin, cyclophosphamide, doxorubicin hydrochloride, doxorubicin hydrochloride liposome (DOXIL®), mirvetuximab soravtansine-gynx (ELAHERE®), gemcitabine hydrochloride (GEMZAR®, INFUGEM®), topotecan hydrochloride (HYCAMTIN®, olaparib (LYNPARZA®), talazoparib (TALZENNA®), niraparib tosylate monohydrate (ZEJULA®), paclitaxel, rucaparib camsylate (RUBRACA®), thiotepa (TEPADINA®), topotecan hydrochloride, abiraterone acetate, cabazitaxel (JEVTANA®), degarelix, enzalutamide (XTANDI®), prednisone, sipuleucel-T (PROVENGE®), or docetaxel.

Additional drugs that may be used to treat cancer include poly(ADP ribose) polymerase (PARP) inhibitors, immune checkpoint inhibitors, and platinum-based agents. PARP inhibitors may include, for example, olaparib, rucaparib, talazoparib, and niraparib. PARP1 is a protein that functions to repair single-stranded nicks in DNA. Drugs that inhibit PARP1 (PARP inhibitors) result in DNA containing multiple double stranded breaks during replication, which can lead to cell death. Immune checkpoint inhibitors work by blocking certain checkpoint proteins from binding with their partner proteins, allowing T cells to kill cancer cells. Immune checkpoint inhibitors may include, for example, pembrolizumab, nivolumab, and cemiplimab. Platinum-based agents are chemical complexes comprising platinum and cause crosslinking of DNA. Crosslinked DNA inhibits DNA repair and synthesis in cancerous cells. Exemplary platinum-based agents may include cisplatin, oxaliplatin, and carboplatin.

### Kits

The polypeptide probes and/or primers or antibodies or polypeptide probes that can be used in the methods described herein can be arranged in a kit. Thus, one embodiment is directed to a kit for diagnosing, prognosing, or predicting the recurrence of cancer, such as basal-like breast cancer, comprising a plurality of polypeptide probes for detecting at least 1, such as at least 2, at least 3, at least 4, at least 5, at least 6, at least 7, at least 8, at least 9, at least 10, at least 11, at least 12, at least 13, at least 14, at least 15, at least 16, or 17 of the phosphopeptides in a 17 phosphopeptide signature, wherein the plurality of polypeptide probes contains polypeptide probes for no more than 500, 250, 100, 75, 60, 50, 40, 30, 20, 15, 10, or 5 polypeptides. In one embodiment, the plurality of polypeptide probes comprises polypeptide probes for detecting all 17 of the aforementioned phosphopeptides.

The kit for diagnosing, prognosing, or predicting recurrence of cancer may also comprise antibodies. Thus, in one embodiment, the kit for diagnosing, prognosing, or predicting recurrence of cancer comprises a plurality of antibodies for detecting at least 1, such as at least 2, at least 3, at least 4, at least 5, at least 6, at least 7, at least 8, at least 9, at least 10, at least 11, at least 12, at least 13, at least 14, at least 15, at least 16, or 17 of the phosphopeptides in the 17 phosphopeptide signature, wherein the plurality of antibodies contains antibodies for no more than 500, 250, 100, 75, 60, 50, 40, 30, 20, 15, 10, or 5 polypeptides. The antibodies may be optionally labeled.

In certain embodiments disclosed herein, there is a kit for use in predicting cancer recurrence and/or prognosing cancer comprising a plurality of probes for detecting a phosphophorylation status of at least 1, such as at least 2, at least 3, at least 4, at least 5, at least 6, at least 7, at least 8, at least 9, at least 10, at least 11, at least 12, at least 13, at least 14, at least 15, at least 16, or 17 of the following phosphorylation sites: a serine at amino acid residue 696 of ARID1A, a threonine at amino acid residue 81 of SGTA, a threonine at amino acid residue 206 of RBM14, a serine at amino acid residue 21 of RAB12, a serine at amino acid residue 275 of ZC3HAV1, a serine at amino acid residue 1070 of CLASP1, or a serine at amino acid residue 886 of EPRS; serine at amino acid residue 339 of KIAA1522, a serine at amino acid residue 280 of RBM14, a serine at amino acid residue 256 of RBM14, a threonine at amino acid residue 498 of PARN, a serine at amino acid residue 14 of PSMD11, a serine at amino acid residue 413 of FOXO3, a serine at amino acid residue 11 of DCK, a serine at amino acid residue 496 of PARN, a serine at amino acid residue 1354 of MYO9B, or a serine at amino acid residue 184 of PLEKHA3. In certain embodiments, the plurality of probes comprises phosphopeptides, including the phosphopeptides described in Tables A and B.

As noted above, the polypeptide probes and antibodies described herein may be optionally labeled with a detectable label. Any detectable label used in conjunction with probe or antibody technology, as known by one of ordinary skill in the art, can be used. As described herein, the labelled polypeptide probes or labelled antibodies are not naturally occurring molecules; that is the combination of the polypeptide probe coupled to the label or the antibody coupled to the label do not exist in nature. In certain embodiments, the probe or antibody is labeled with a detectable label selected from the group consisting of a fluorescent label, a chemiluminescent label, a quencher, a radioactive label, biotin, mass tags and/or gold.

In one embodiment, a kit includes instructional materials disclosing methods of use of the kit contents in a disclosed method. The instructional materials may be provided in any number of forms, including, but not limited to, written form (e.g., hardcopy paper, etc.), in an electronic form (e.g., computer diskette or compact disk) or may be visual (e.g., video files). The kits may also include additional components to facilitate the particular application for which the kit is designed. Thus, for example, the kits may additionally include other reagents routinely used for the practice of a particular method, including, but not limited to buffers, enzymes, labeling compounds, and the like. Such kits and appropriate contents are well known to those of skill in the art. The kit can also include a reference or control sample. The reference or control sample can be a biological sample or a data base.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example 1

Sample collection: Fresh breast tissue specimens were collected from patients following excisional biopsy from 2001 to 2010. After undergoing gross pathology assessment, breast tissue specimens were embedded in Optimal Cutting Temperature (OCT) compound, quick-frozen, and stored at −180° C. in liquid nitrogen freezers.

Immunohistochemistry (IHC) subtyping was used to enrich the cohort with non-luminal A (LA) tumors. The IHC subtypes for 117 primary breast cancer tissue samples were determined using IHC assays for ER, PR, HER2, and Ki67 in a centralized CLIA-certified laboratory following standardized protocols. The study cohort included 30 triple negative (TN; ER−/PR−/HER2−), 16 HER2+ (ER−/PR−/HER2+), 39 Luminal B1 (LB1; ER+/HER2+/Ki67+), 17 Luminal B2 (LB2; ER+/HER2+), and 15 Luminal A (LA; ER+/HER2−/Ki67−) subtypes.

Laser microdissection and molecular extraction: OCT-embedded breast tumors were processed by laser microdissection (LMD) to collect and enrich for tumor cells. OCT-embeded specimens were sectioned at 8 μm inside a temperature-controlled cryostate (Leica Microsystems, Buffalo Grove, IL) and mounted on polyethylene-naphthalate (PEN) membrane slides (W. Nuhsbaum Inc., McHenry, IL). Scout slides were created by mounting every 10^{th} section on microscopic plus slides and staining with hematoxylin and eosin, and regions of interest for LMD were marked by a pathologist. Next, PEN membrane slides were stained with cresyl violet staining solution (Ambion/Applied Biosystems, Grand Island, NY), and LMD performed according to the marked regions of interest using the Leica ASLMD system. Following LMD, the collected sample was incubated for 10 minutes at 37° C. in an air incubator. After incubation, the sample was vortexed briefly, a quick spin performed, and the sample pipetted up and down several times before transferring the lysate to a DNA column.

DNA, RNA, and protein were then simultaneously extracted from each tumor specimen using the Illustra triplePrep kit (Cytiva, Marlborough, MA) following the manufacturer's protocol. The optional DNase treatment of the RNA was performed. Protein pellets were washed 2-3 times with 1 mL of nuclease-free water and then re-suspended in 100 μl of 8M urea in 100 mM ammonium bicarbonate, pH 7.8. Following isolation with the triplePrep kit, the tumor DNA samples were further cleaned using the Genomic DNA Clean & Concentrator-10 kit (Zymo Research Corporation, Irvine, CA) to remove protein contaminants. The concentrations of the DNA, RNA and protein samples were measured using the Qubit fluorometer (Thermo Fisher Scientific Inc., Waltham, MA), and the integrity of the RNA samples was determined using the Bioanalyzer (Agilent Technologies, Inc., Santa Clara, CA). Germline blood DNA (“normal”) from clots was extracted from BD Vacutainer 10 mL serum collection tubes (Becton, Dickinson and Company, Franklin Lakes, NJ) using the Gentra Puregene Blood Kit (Qiagen Sciences, Germantown, MD), and the concentrations measured using the Qubit fluorometer. Tumor DNA and germline DNA samples were normalized to final concentrations of 5 ng/μl in a total volume of 100 μl and 10 ng/μl in a total volume of 50 μl, respectively, for whole genome sequencing (WGS). RNA samples were diluted to 50 ng/μl for total RNA sequencing (RNA-Seq).

Tryptic digestion of proteins: Approximately 400 μg of proteins from 100 μL of each sample were diluted and re-suspended using 300 μL of lysis buffer (8 M urea, 100 mM NH₄HCO₃, pH 8.0, 10 mM NaF, phosphatase inhibitor cocktail 2, phosphatase inhibitor cocktail 3, 20 μM PUGNAc). Lysates were pre-cleared by centrifugation at 16,500 g for 5 minutes at 4° C., and protein concentrations were determined by BCA assay (Pierce). Proteins were reduced with 5 mM dithiothreitol for 1 hour at 37° C. and subsequently alkylated with 10 mM iodoacetamide for 1 hour at 25° C. in the dark. Samples were diluted 1:2 with 100 mM NH₄HCO₃ and 1 mM CaCl₂ and digested with sequencing-grade modified trypsin (Promega) at 1:50 enzyme-to-substrate ratio. After 4 hours of digestion at 37° C., samples were diluted 1:4 with the same buffers, and another aliquot of the same amount of trypsin was added to the samples and further incubated at 25° C. overnight (16 hours). The digested samples were then acidified with 10% trifluoroacetic acid to a pHl of about 3. Tryptic peptides were desalted on strong cation exchange SPE (Supelco) and reversed-phase C18 SPE columns (Supelco) and dried using a Speed-Vac.

TMT-6 Labeling: The desalted peptides from each sample were labeled with 6-plex Tandem Mass Tag (TMT) reagents according to the manufacturer's instructions (ThermoScientific). Peptides (100 μg) from each of the samples were dissolved in 30 μL of 500 mM triethylammonium bicarbonate, pH 8.5, and mixed with one unit of TMT reagent that was dissolved freshly in 70 μL of anhydrous acetonitrile. Channel 131 was used for labeling the pooled internal reference sample (pooled from all tumor samples with equal contribution) throughout the sample analysis. After a 1 hour incubation at room temperature, 8 μL of 5% hydroxylamine was added and incubated for 15 minutes at room temperature to quench the reaction. Peptides labeled by different TMT reagents were then mixed, dried down to about 250 μL using a Speed-Vac, and desalted on C18 SPE columns.

Peptide fractionation by basic reversed-phase liquid chromatography: Approximately 400 μg of 6-plex TMT-labeled sample was separated on a Waters reversed-phase XBridge C18 column (250 mm×4.6 mm column containing 5-μm particles and a 4.6 mm×20 mm guard column) using an Agilent 1200 HPLC System. After sample loading, the C18 column was washed for 35 minutes with solvent A (10 mM ammonium formate, pH 7.5), before applying a 112-min LC gradient with solvent B (10 mM ammonium formate, pH 7.5, 90% acetonitrile). The LC gradient began with a linear increase of solvent A to 10% B in 6 minutes, then linearly increased to 30% B in 86 minutes, 10 minutes to 42.5% B, 5 minutes to 55% B, and 5 minutes to 100% B. The gradient then resolved to 100% solvent A in 1 minute and waskept at 100% solvent A for 30 minutes. The flow rate was 0.5 mL/min. A total of 96 fractions were collected from 48 to 164 minutes of the LC gradient into a 96-well plate (1.2 mL per fraction), Fractions 1-75 were concatenated into 12 fractions by combining the fractions that were 13 fractions apart; fractions 76-96 were pooled as a 13th fraction. For proteome analysis, 5% of each of the 12 concatenated fractions was dried and re-suspended in 2% acetonitrile and 0.1% formic acid to a peptide concentration of 0.1 μg/μL for LC-MS/MS analysis. The remainder of the 12 concatenated fractions (95%) were further concatenated into six fractions by combining two concatenated fractions (i.e., combining concatenated fractions #1 and #7; #2 and #8; and so on), dried, and subjected to immobilized metal affinity chromatography (IMAC) for phosphopeptide enrichment. The 13th fraction was not split and combined further, like the other fractions, and it was subjected to IMAC enrichment directly; the resulting eluant was analyzed as the 7th phosphoproteome fraction, and the IMAC flow-through was analyzed as the 13th global proteome fraction.

Phosphopeptide enrichment using IMAC: Fe³⁺-NTA-agarose beads were freshly prepared using Ni-NTA magnetic agarose beads (QIAGEN) for phosphopeptide enrichment. For each of the six fractions from the same TMT-6 plex, peptides were reconstituted in 135 μL IMAC binding/wash buffer (80% acetonitrile, 0.1% TEA) and incubated with end-over-end rotation with 35 μL of the 50% bead suspension for 30 minutes at room temperature. After incubation, the beads were washed four times each with 150 μL of wash buffer. Phosphopeptides were eluted from the beads using 50 μL of elution buffer (1:1 acetonitrile: 5% ammonia water in 5 mM pH 8 phosphate buffer, pH˜10), and acidified immediately to pH 3.5-4 with 10% TFA. Samples were dried using a Speed-Vac and later reconstituted with 20 μL of 3% acetonitrile, 0.1% formic acid for LC-MS/MS analysis.

LC-MS/MS analysis: The global proteome and phosphoproteome fractions were separated using a Waters nano-Acquity dual pumping UPLC system (Milford, MA) custom configured for on-line trapping of a 10-μL injection at 3 μL/min with reverse direction elution onto the analytical column at 300 nL/min. Columns were packed using 360-μm o.d. fused silica (Polymicro Technologies Inc., Phoenix, AZ) with 5-mm sol-gel frits for media retention and contained Jupiter C18 media (Phenomenex, Torrence, CA) in 5-μm particle size for the trapping column (150 μm i.d.×4 cm long) and 3-μm particle size for the analytical column (75 μm i.d.×70 cm long). Mobile phases consisted of (A) 0.1% formic acid in water and (B) 0.1% formic acid in acetonitrile with the following gradient profile (min, % B): 0, 1; 2, 8; 20, 12; 75, 30; 97, 45; 100, 95; 110, 95; 115, 1; 150, 1.

MS analysis was performed using a Q-Exactive Plus mass spectrometer (Thermo Scientific, San Jose, CA) outfitted with a nano-electrospray ionization interface. Electrospray emitters were prepared using 150 μm o.d.×20 μm i.d. chemically etched fused silica. The heated capillary temperature and spray voltage were 325° C. and 2.3 kV, respectively. Data were collected for 100 minutes following a 15 minutes delay from sample injection. Orbitrap precursor spectra (AGC 1×10⁶) were collected from 400 to 2000 m/z at a resolution of 35,000 with the top-ten data-dependent Orbitrap HCD MS/MS spectra at a resolution of 17,500 (AGC 1×10⁵) and max ion time of 100 ms. Masses selected for MS/MS were isolated at a width of 2.0 m/z and fragmented using a normalized collision energy of 30% and a dynamic exclusion time of 30 seconds.

Proteomics data processing: The Thermo RAW files were converted to mzML format using the msConvert tool in Proteo Wizard. These files were used to search against the reference proteome hg 19 from Ensembl release 75. The partially tryptic search used a #10 ppm parent ion tolerance, allowed for isotopic error in precursor ion selection, and searched a decoy database composed of the forward and reverse protein sequences. MS-GF+considered static carbamidomethylation (+57.0215 Da) on cysteine residues, TMT modifications (+229.1629 Da) on peptide N termini and lysine residues, and dynamic oxidation (+15.9949 Da) on methionine residues for searching the global proteome data. Peptide identification stringency was set to a maximum FDR of 1% at the peptide level using PepQValue <0.005 and parent ion mass deviation <8 ppm criteria. A minimum of 6 unique peptides per 1000 amino acids of protein length was required for achieving 1% at the protein level within the full dataset. Inference of the parsimonious protein set resulted in the identification of a total of 8,019 common protein groups among the 112 samples. Phosphopeptides were identified from the phosphoproteomics data files as described above (e.g., peptide level FDR<1%), with an additional dynamic phosphorylation (+79.9663 Da) on serine, threonine, or tyrosine residues. The phosphoproteome data were further processed by the Ascore algorithm for phosphorylation site localization, and the top-scoring sequences were reported. Prioritized protein inference (proteins that passed inference in global) was kept and shared peptides were dropped.

The intensities of all six TMT reporter ions were extracted using MASIC software. Next, PSMs were linked to the extracted reporter ion intensities by scan number. The reporter ion intensities from different scans and different fractions corresponding to the same protein or phosphopeptide were summed. Relative protein or phosphopeptide abundance was calculated as the ratio of abundance in a given sample to the reference abundance. The pooled reference sample was labeled with TMT 131 reagent, allowing comparison of relative protein or phosphopeptide abundances across different TMT-6 plexes. The relative abundances were log 2 transformed and zero-centered for each protein and phosphopeptide to obtain final, relative abundance values. Sample quality control of the quantified proteins was performed using a density plot, which demonstrated that all samples conformed to an expected unimodal distribution. Principal component analyses (PCA) were performed to confirm that there were no sequencing batch effects after normalization.

Proteome and phosphoproteome clusters: Robust clusters were derived with consistently detected proteins and phosphopeptides in all tumors for proteomics and phosphoproteomics, respectively. In the case of phosphoproteomic data, there were 331 consistently detected phosphopeptides associated with 245 unique genes and 245 unique proteins. From the many phosphopeptides for a gene, the one with the highest variation based on the standard deviation metric was selected. For proteomic data, there were 1461 consistently detected proteins, corresponding to 1461 unique proteins and 1457 unique genes. From the many proteins for a gene, the one with the highest variation based on the standard deviation metric was selected. These values were median centralized and used for clustering. Consensus clustering was performed using the ConsensusClusterPlus R Bioconductor package. The features were transformed into 1000 bootstrap sample data sets with a probability of 0.8 for selecting any sample and any protein. The bootstrap data sets were clustered using k-means clustering with up to 6 clusters, Based on both visual inspection of the consensus matrix and the silhouette plots for identifying better coherence, the clusters were selected.

Mertins et al. 2016 dataset: The proteome and phosphoproteome dataset from this study was obtained from the supplemental data of Mertins et al., Proteogenomics connects somatic mutations to signaling in breast cancer, Nature 2016, 534(7605):55-62, as well as through personal communication with the corresponding author of the study. The data from the ESTIMATE scores of the proteome clusters of this study was then compared with those of the Mertins et al. 2016 paper.

Protein-mRNA correlation: Gene-wise Pearson correlation coefficients were calculated for each mRNA and protein pair, including mRNA from RNA-Seq and protein from global proteomics, across the cohort. Sample-wise Pearson correlation coefficients were calculated for each sample's mRNA and protein features. To derive the correlation for the Mertins et al. 2016 study, protein data was obtained from the supplemental files, and the relevant RNA-Seq data was taken from the TCGA-BRCA dataset as previously mentioned. Correlation coefficients and FDR adjusted p-values were calculated in R.

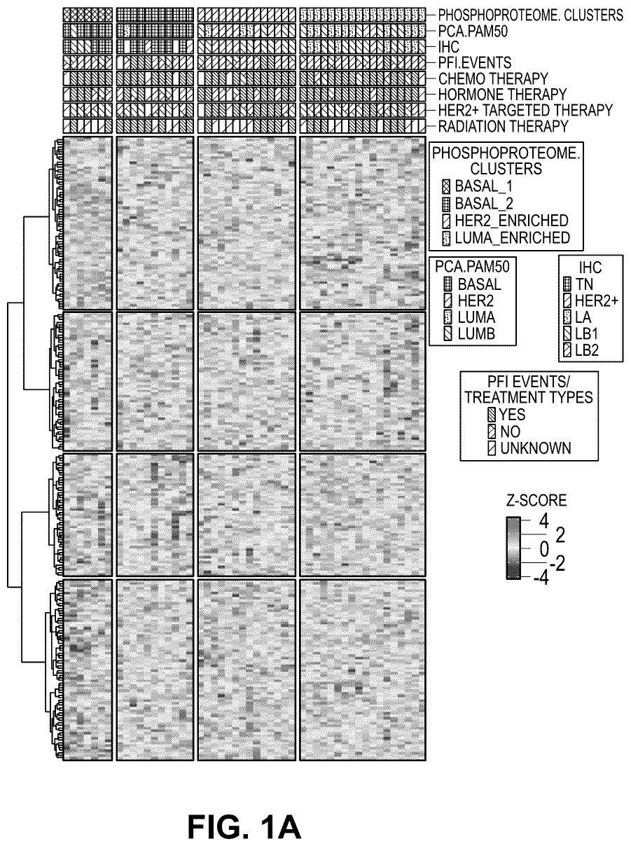

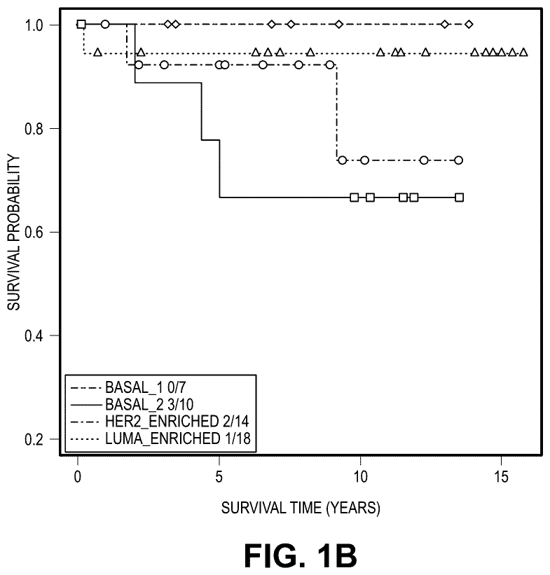

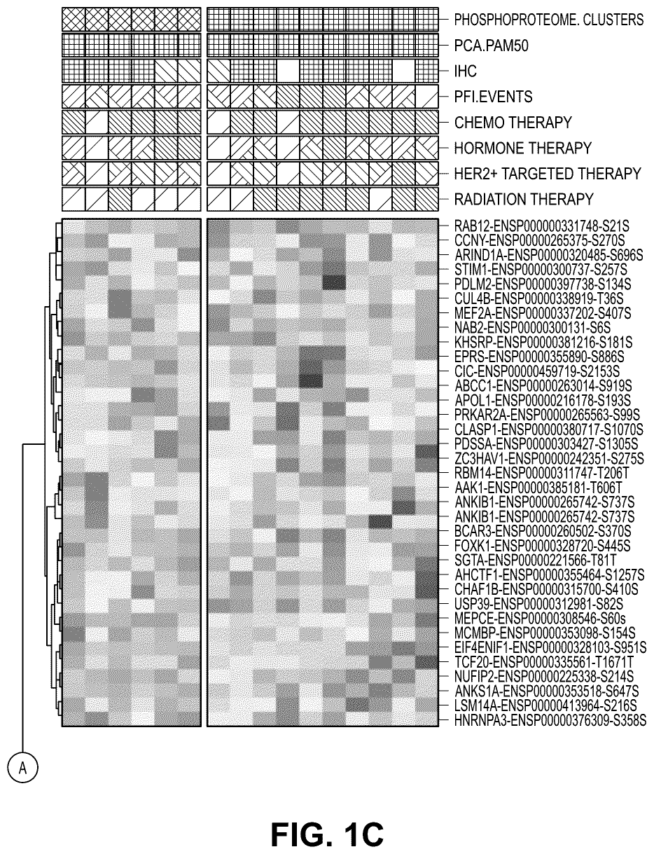

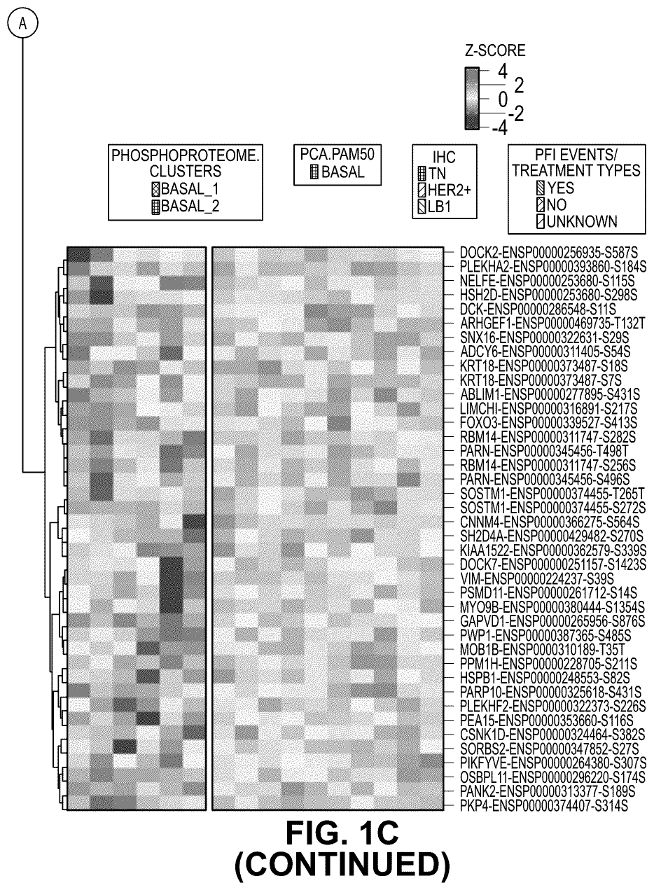

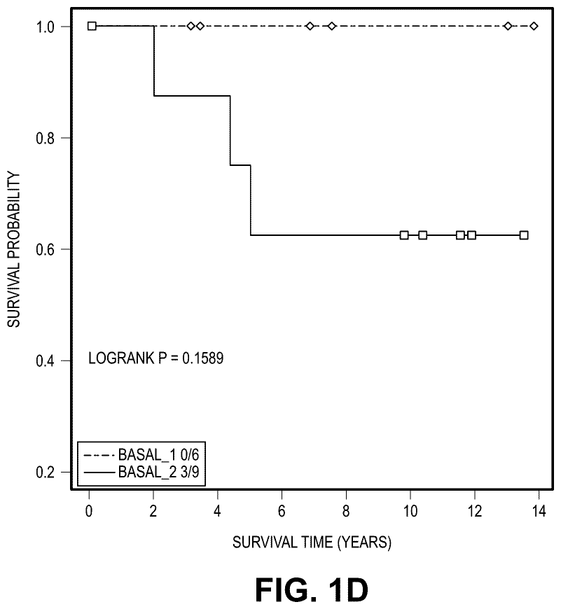

Results: The phosphoproteomic clustering analysis revealed basal clusters with trended outcome differences. MS-based global phosphoproteomics quantified (FDR<0.01) a total of 5,049 phosphopeptides (from 2,093 proteins and 2,065 genes) in at least one of 50 cases. The 331 phosphopeptides (from 245 genes) quantified in all of the cases for phosphoproteomics were used for differential expression analyses. For clustering analysis, to minimize any potential bias from genes with multiple peptides, the phosphopeptide per gene with the highest variation was selected, resulting in 245 phosphopeptides. Unsupervised K-Means consensus clustering using these 245 unique phosphopeptides resulted in 4 optimal clusters (FIG. 1), including two Basal-enriched clusters designated as Basal 1 (n=7, 85.7% Basal) and Basal 2 (n=11, 90.9% Basal), a Her2-enriched cluster (n=14, 50% Her2), and a LumA-enriched cluster (n=18, 55% LumA) (FIG. 1A). Survival analyses of the 4 clusters were performed using the endpoint of PFI, and it was observed that although not statistically significant, the Basal 2 cluster had the worst survival, and surprisingly, the Basal 1 cluster had no PFI events (FIG. 1B).

The differences among the Basal cases were then examined in more detail. The 10 Basal cases in the Basal-2 cluster were identified as the high relapse-risk group, and the 6 cases in the Basal-1 cluster were identified as the low relapse-risk group. The differential expression analysis between the two groups, with all of the 331 quantified phosphopeptides, identified 40 and 36 significantly (FC>1.2 for upregulated, FC<1/1.2 for downregulated, and FDR≤0.2) up-regulated and down-regulated phosphopeptides, respectively, in the Basal-2 versus Basal-1 clusters. The unsupervised hierarchical clustering of the tumors using these 76 phosphopeptides captured the distinct profiles of the two Basal groups (FIG. 1C), There was also a trending PFI difference between the two Basal clusters (p=0.16; FIG. 1D).

To explore potential markers of survival outcome differences, each of the 76 phosphopeptides was tested, using median separation of expression, for its ability to separate Basal cases into high relapse-risk and low relapse-risk groups. Most of the 76 differentially expressed phosphopeptides, by their high (>median) and low expression (≤median), provided at least a trending separation of high relapse-risk and low relapse-risk Basal cases.

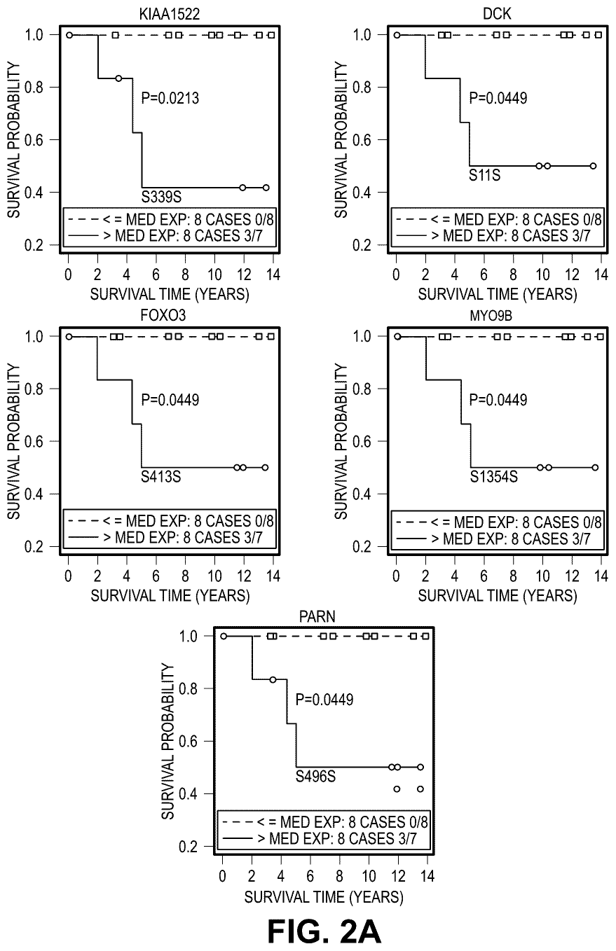

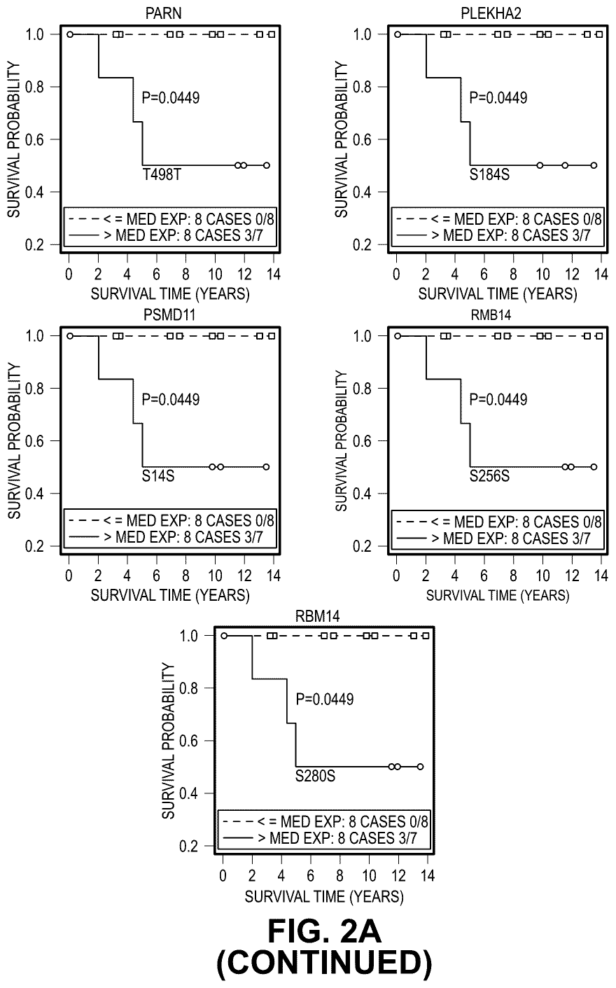

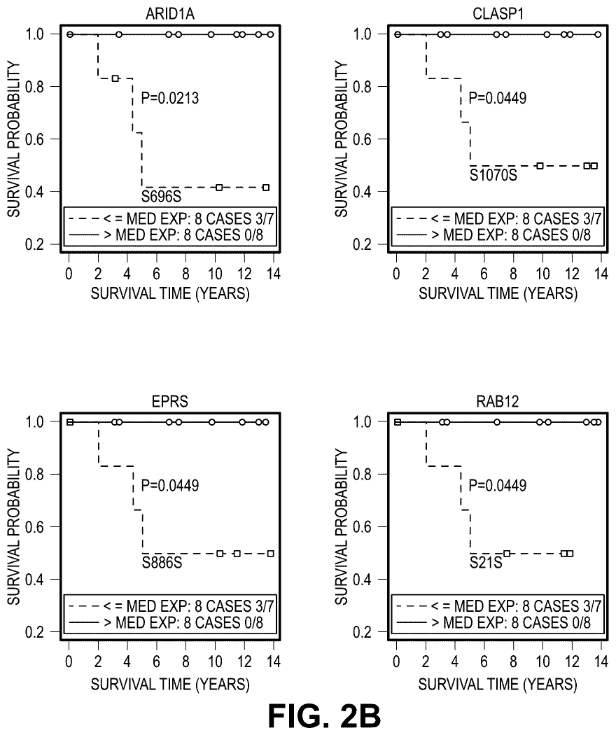

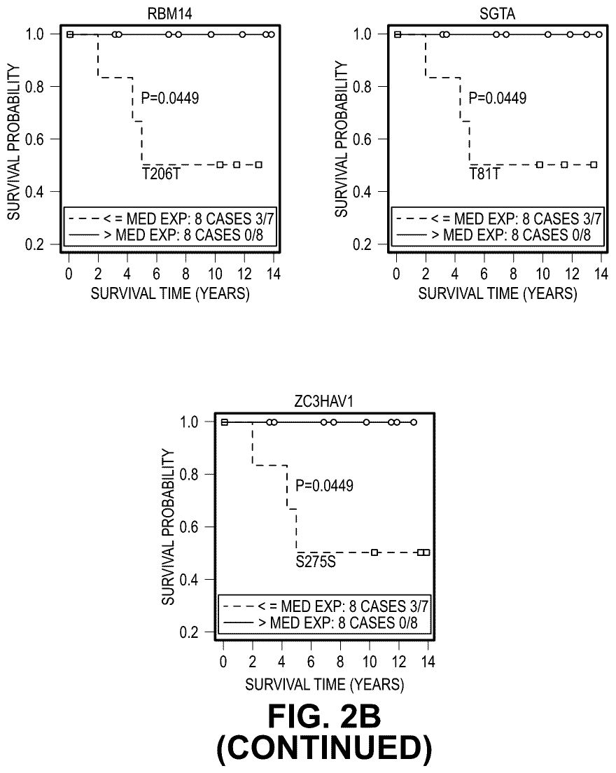

Seventeen phosphopeptides were able to significantly distinguish high relapse-risk cases from the low relapse-risk cases (log rank p<0.05; FIG. 2 and Table 1). Of the seventeen phosphopeptides, 10 were up-regulated (FIG. 2A) and 7 were down-regulated (FIG. 2B), representing 14 genes. Notably, among the 17 phosphopeptides, three were from the gene RBM14, two of which were up-regulated (sites S280s and S256s) and one that was down-regulated (site T206t) in the high relapse-risk group. Many of the 14 genes represented by the 17 phosphopeptides identified bere have been previously reported to play significant roles in breast cancer, including KIAA1522, DCK, FOXO3, and MYO9B among the up-regulated genes and ARID1A, EPRS, and ZC3HAV1 among the down-regulated genes in the high relapse-risk cases.

To further investigate whether any outcome differences may be influenced by the different treatments the patients received, the cases shown in FIG. 1 were annotated with types of treatments. As shown in FIG. 1C, there were no observed treatment differences among the Basal cases in the two different groups (Fisher exact test p=1.0).

Tumor samples from the 16 basal-like breast cancer patients in the Basal 1 and Basal 2 clusters were evaluated, and a recurrence index was calculated using Formula 1 as described herein. A scaled recurrence index score was further calculated using Formula 2 as described herein. Based on the calculated scaled recurrence index score, the patients were further categorized as being at a low risk for cancer recurrence (RI.group=RI.low in Table 1 below) or a high risk for cancer recurrence (RI.group=RI.high in Table 1 below). The results are shown in Table 1 below. The patients were further categorized into three groups based on three cutoff methods, as follows: RI.group 1 indicates a clustering-based cutoff; RI.group 2 indicates a turn of the RI sign cutoff; and RI.group 3 indicates a score next to the PFI event cutoff.

TABLE 1
Recurrence Index calculated for 16 basal-like breast cancer patients

| | | | | | | | Phospho- | | PFI | PFI |
|---|---|---|---|---|---|---|---|---|---|---|
| | | | | | | | proteome | | time | time |
| Patient_ID | RI | RI.scaled | RI.group 1 | RI.group 2 | RI.group 3 | PCA.PAM50 | Clusters | PFI | (days) | (years) |
| Patient_1 | 39.46 | 10.00 | RI high | RI high | RI high | Basal | Basal_2 | 1 | 738 | 2.02 |
| Patient_2 | 32.26 | 9.16 | RI high | RI high | RI high | Basal | Basal_2 | 1 | 1604 | 4.39 |
| Patient_3 | 18.39 | 7.53 | RI high | RI high | RI high | Basal | Basal_2 | | Not | |
| | | | | | | | | | available | |
| Patient_4 | 16.06 | 7.25 | RI high | RI high | RI high | Basal | Basal_2 | 0 | 34 | 0.09 |
| Patient_5 | 15.22 | 7.15 | RI high | RI high | RI high | Basal | Basal_2 | 0 | 4216 | 11.54 |
| Patient_6 | 14.18 | 7.03 | RI high | RI high | RI high | Basal | Basal_2 | 1 | 1835 | 5.02 |
| Patient_7 | 8.99 | 6.42 | RI high | RI high | RI low | Basal | Basal_2 | 0 | 4939 | 13.52 |
| Patient_8 | −3.74 | 4.93 | RI high | RI low | RI low | Basal | Basal_2 | 0 | 4347 | 11.90 |
| Patient_9 | −7.00 | 4.54 | RI high | RI low | RI low | Basal | Basal_2 | 0 | 3786 | 10.37 |
| Patient_10 | −9.45 | 4.26 | RI high | RI low | RI low | Basal | Basal_2 | 0 | 3579 | 9.80 |
| Patient_11 | −23.11 | 2.65 | RI low | RI low | RI low | Basal | Basal_1 | 0 | 1260 | 3.45 |
| Patient_12 | −26.39 | 2.27 | RI low | RI low | RI low | Basal | Basal_1 | 0 | 2753 | 7.54 |
| Patient_13 | −33.05 | 1.49 | RI low | RI low | RI low | Basal | Basal_1 | 0 | 5059 | 13.85 |
| Patient_14 | −40.32 | 0.63 | RI low | RI low | RI low | Basal | Basal_1 | 0 | 2513 | 6.88 |
| Patient_15 | −40.45 | 0.62 | RI low | RI low | RI low | Basal | Basal_1 | 0 | 4760 | 13.03 |
| Patient_16 | −45.70 | 0.00 | RI low | RI low | RI low | Basal | Basal_1 | 0 | 1165 | 3.19 |

As shown in Table 1, for the RI.group 1 clustering-based cutoff, 9 high relapse risk cases had 3 PFI events (true positives), and 6 high relapse risk cases that did not have a PFI event (false positive), while 6 low relapse risk cases had 0 PFI events (true negatives). There were zero false positives in the low relapse risk cases (p=0.1589). The RI.group 1 clustering based cutoff had a sensitivity of 100% and specificity of 50%.

For the RI.group 2, there were 6 high relapse risk cases for which PFI data was available, wherein 3 had a PFI event (true positive) and 3 did not have a PFI event (false positive), while there were 9 low relapse risk cases, wherein 0 had a PFI event (true negative), such that there were no false negatives in the RI.group.2 (p=0.0157). The RI.group 2 turn of the RI sign cutoff had a sensitivity of 100% and specificity of 75%.

For the RI. group 3, there were 5 high risk relapse cases for which PFI data was available, wherein 3 had a PFI event (true positive) and 2 did not have a PFI event (false positive), while there were 10 low relapse risk cases, wherein 0 had a PFI event (true negative), such that there were no false negatives in the RI.group 3 (p=0.0028). The RI.group 3 score next to PFI even cutoff had a sensitivity of 100% and a specificity of 83%.

All patents, patent applications, and published references cited herein are hereby incorporated by reference in their entirety. While this disclosure has been particularly shown and described with references to preferred embodiments thereof, it will be understood by those skilled in the art that various changes in form and details may be made therein without departing from the scope of the disclosure encompassed by the appended claims.

1. A method of predicting cancer recurrence in a patient, comprising:
(a) determining the phosphorylation status of at least one protein in a biological sample obtained from the patient, wherein the at least one protein is selected from ARID1A, SGTA, RBM14, RAB12, ZC3HAV1, CLASP1, EPRS, KIAA1522, PARN, PSMD11, FOXO3, DCK, MYO9B, or PLEKHA2;
(b) identifying the patient as having a high risk of cancer recurrence if the phosphorylation status of the at least one protein is
(i) under-phosphorylated as compared to a control in at least one of the following locations: a serine at amino acid residue 696 of ARID1A, a threonine at amino acid residue 81 of SGTA, a threonine at amino acid residue 206 of RBM14, a serine at amino acid residue 21 of RAB12, a serine at amino acid residue 275 of ZC3HAV1, a serine at amino acid residue 1070 of CLASP1, or a serine at amino acid residue 886 of EPRS; or
(ii) over-phosphorylated as compared to a control in at least one of the following locations: a serine at amino acid residue 339 of KIAA1522, a serine at amino acid residue 280 of RBM14, a serine at amino acid residue 256 of RBM14, a threonine at amino acid residue 498 of PARN, a serine at amino acid residue 14 of PSMD11, a serine at amino acid residue 413 of FOXO3, a serine at amino acid residue 11 of DCK, a serine at amino acid residue 496 of PARN, a serine at amino acid residue 1354 of MYO9B, or a serine at amino acid residue 184 of PLEKHA3; and
(c) optionally administering to the patient a therapeutically effective amount of a cancer therapy if the patient is classified as having a high risk of cancer recurrence.

2. The method of claim 1, wherein the cancer is breast cancer.

3. The method of claim 2, wherein the breast cancer is a basal-like breast cancer.

4. The method of claim 1, wherein the phosphorylation status of at least two, at least three, at least four, at least five, at least six, at least seven, at least eight, at least nine, at least ten, at least eleven, at least twelve, at least thirteen, or all fourteen of the proteins is determined.

5. The method of claim 1, wherein the at least one protein is selected from ARID1A, SGTA, RBM14, RAB12, ZC3HAV1, CLASP1, or EPRS, and at least one, at least two, at least three, at least four, at least five, at least six, or seven of the proteins are under-phosphorylated as compared to a control.

6. The method of claim 1, wherein the at least one protein is selected from KIAA1522, RBM14, PARN, PSMD11, FOXO3, DCK, MYO9B, or PLEKHA3, and at least one, at least two, at least three, at least four, at least five, at least six, at least seven, or eight of the proteins are over-phosphorylated as compared to a control.

7. The method of claim 1, further comprising obtaining from the patient a biological sample comprising cancer tissues or cells.

8. A method of treating a patient, comprising administering to the patient a therapeutically effective amount of a cancer therapy, wherein the patient has been identified as having a high risk of cancer recurrence according to the method of claim 1.

9. The method of claim 8, wherein the cancer therapy is one or more of surgery, radiation therapy, hormone therapy, chemotherapy, biological therapy, or high intensity focused ultrasound.

10. The method of claim 1, wherein the control comprises control tissues or cells or the phosphorylation status obtained from control tissues or cells.

11. The method of claim 10, wherein the control tissues or cells are obtained from a patient or pool of patients who exhibited non-recurrent cancer or non-cancerous tissues or cells.

12. The method of claim 10, wherein the control comprises a standard or reference that reflects the phosphorylation status of phosphopeptides in a sample or pool of samples known to contain non-recurrent cancer or known to be cancer-free.

13. The method of claim 1, wherein the phosphorylation status of a phosphopeptide signature is determined by calculating a recurrence index score for the phosphopeptide signature.

14. The method of claim 13, wherein the recurrence index is calculated as the sum of the weights calculated for each phosphopeptide in the phosphopeptide signature.

15. The method of claim 14, wherein the recurrence index is calculated using Formula 1:
$$R I = {\sum }_{i = 1}^{17} {w}_{i} {log}_{2} {x}_{i} \qquad Formula 1$$
wherein the raw recurrence index (RI) is calculated by the weighted linear combination of log 2 transformed normalized assay results of phosphopeptides x, with t-statistic w, for each phosphopeptide serving as the weight.

16. The method of claim 15, further comprising scaling the raw recurrence index score to 0-10 using a transformation formula as set forth in Formula 2:
$$R I . {scale}_{i} = \frac{max ( R I ) - R {I}_{i}}{max ( R I ) - min ( R I )} \times 10 \qquad Formula 2$$
to calculate a scaled recurrence index.

17. The method according to claim 16, wherein the if the scaled recurrent index is above a threshold value (e.g., 3, 4, 5, 6, or 7), the patient is classified as having a high risk of cancer recurrence.

18. The method according to claim 1, wherein the phosphorylation status of the at least one protein is determined by using immunohistochemical (IHC), protein array, or mass spectrometry (MS)-based technologies.

19. A kit for use in predicting cancer recurrence and/or prognosing cancer, the kit comprising a plurality of probes for detecting a phosphophorylation status of at least 1, such as at least 2, at least 3, at least 4, at least 5, at least 6, at least 7, at least 8, at least 9, at least 10, at least 11, at least 12, at least 13, at least 14, at least 15, at least 16, or 17 of the following phosphorylation sites: a serine at amino acid residue 696 of ARID1A, a threonine at amino acid residue 81 of SGTA, a threonine at amino acid residue 206 of RBM14, a serine at amino acid residue 21 of RAB12, a serine at amino acid residue 275 of ZC3HAV1, a serine at amino acid residue 1070 of CLASP1, or a serine at amino acid residue 886 of EPRS; serine at amino acid residue 339 of KIAA1522, a serine at amino acid residue 280 of RBM14, a serine at amino acid residue 256 of RBM14, a threonine at amino acid residue 498 of PARN, a serine at amino acid residue 14 of PSMD11, a serine at amino acid residue 413 of FOXO3, a serine at amino acid residue 11 of DCK, a serine at amino acid residue 496 of PARN, a serine at amino acid residue 1354 of MYO9B, or a serine at amino acid residue 184 of PLEKHA3, wherein the plurality of probes contains probes for detecting no more than 500 different phosphopeptides.
